(19) United States
(12) Patent Application Publication
(10) Pub. No.: US 2023/0286844 A1
(43) Pub. Date: Sep. 14, 2023

(54) METHOD FOR DECONTAMINATING ENVIRONMENT POLLUTED WITH PETROLEUM-RELATED MATERIAL, AND MATERIAL USED

(71) Applicant: IGA BIO RESEARCH CO., LTD., Sakai-shi, Osaka (JP)

(72) Inventor: Takuo SAKAI, Sakai-shi, Osaka (JP)

(21) Appl. No.: 17/261,426

(22) PCT Filed: Jul. 2, 2019
(86) PCT No.: PCT/JP2019/026248

§ 371 Date: Jan. 19, 2021

(30) Foreign Application Priority Data
Jul. 6, 2018 (JP) 2018-128764

(51) Int. Cl. C02F 3/34; C12N 1/26; C12N 1/20; B09C 1/10

(52) U.S. Cl. CPC C02F 3/341; B09C 1/10; C02F 3/344; C02F 3/348; C12N 1/26; C12N 1/205; C12R 2001/07

(57) ABSTRACT

Techniques of decontaminating environments polluted with petroleum-related materials through the use of microbes have problems associated with difficulty in construction that requires high-pressure injection deep in the ground, due to low pressure resistance of any of the conventional microbes. In many cases, the survival of the microbes is influenced by temperatures, etc. of the environments and disadvantageously requires extreme caution in conditions of preservation or the environments (temperature, pressure, pH, etc.) for use. A microbe having resistance to even high temperatures or high pressures while having excellent ability to degrade petroleum-related materials has been obtained. As a result, the present invention provides a bioremediation technique, particularly, a bioaugmentation technique, which employs an organism resistant to environmental loads such as temperature, pH, and pressure, is highly useful, and utilizes the excellent ability to degrade petroleum-related materials.

### INCORPORATION BY REFERENCE

The present application identifies the name of the ASCII text file name, “TCP-044-Sequential-listing,” the date of creation, “Aug. 4, 2021,” and the size of the ASCII text file in bytes,“1.88 KB,” the disclosures of which are hereby incorporated by reference of the material in the ASCII text file.

### TECHNICAL FIELD

The present invention relates to the decontamination of soil or/and water polluted with petroleum-related materials using a microbe.

### BACKGROUND ART

As redevelopment projects of land and legislating of soil pollution control measures have been pursued in countries across the world in recent years, there has been a demand for safe and inexpensive approaches of purifying land or groundwater polluted with harmful materials. Measures to purify soil or environmental water quality have been actively studied since the 1990s both at home and abroad, and not a few methods have been put in practical use. However, most of these methods are based on physical treatment methods such as washing, heat treatment, or removal by suction, or chemical treatment methods such as degradation and removal or insolubilization using chemicals.

Meanwhile, against a backdrop of the progression of research on microbes degrading petroleum components, a technique of exploiting, for repair, microbes that reside at pollution sites (biostimulation) has been developed and is under practical use. However, the biostimulation has the disadvantage that decontamination is limited by the biological functions of the residing microbes. Now, the development of bioaugmentation, which is a method of introducing microbes degrading pollutants into polluted soil, and maintaining high concentrations is raising expectations as an approach of efficiently decontaminating pollutants by introducing microbes completely degrading the pollutants [Non Patent Literature 1]. The Ministry of Economy, Trade and Industry and the Ministry of the Environment of Japan announced “Guidelines on Use of Microbial-based Bioremediation” in March 2005 as guidelines for purification by introducing microbes into soil or to groundwater pollution sites [Non Patent Literature 2]. The guidelines have not yet been widely distributed, though the systems officially certify the safety of the microbes used and decontamination techniques and methods.

The bioaugmentation requires selectively activating useful microbes in natural environments and thus often injects aqueous solutions containing microbes by pressurization.

Petroleum-degrading microorganisms including bacteria of the genera Pseudomonas, Acinetobactor, Rhodococcus, and Acinetobacter and yeasts of the genera Candida and Rhodotorula have been isolated so far as practical microbes that can be utilized in the bioremediation of petroleum pollution [Non Patent Literature 3]. Microbes that have previously been used for the purpose of injecting the microbes having the ability to degrade petroleum to polluted environments, and degrading pollutants for decontamination have low degradative activity against macromolecular (having 30 or more carbon atoms) hydrocarbons, and their cells die by high pressures. Therefore, there are problems to be technically solved for injection deep in the ground.

In general, microbial cells die when pressurized. Therefore, bioremediation in deep environments, which inevitably require injecting microbes by pressurization, such as environments in soil is very difficult. Thus, the development of novel techniques is very significant. Meanwhile, there is a report on the activation and disinfection of endospores of the genus Bacillus by pressure [Non Patent Literature 4].

### CITATION LIST

### Non Patent Literature

Non Patent Literature 1: Yoh Takahata: Possibility and problem for practical application of bioaugmentation, Journal of Environmental Biotechnology, Vol. 13, 19-23, 2013
Non Patent Literature 2: Guidelines on Use of Microbial-based Bioremediation: March 2005, the Ministry of Economy, Trade and Industry and the Ministry of the Environment of Japan
Non Patent Literature 3: What microbes degrade petroleum?: National Institute of Technology and Evaluation (Biotechnology Center Homepage https://www.nite.go.jp/nbrc/industry/bioreme2009/knowledg e/bacteria/bacteria-1.html)
Non Patent Literature 4: Kenji Nishi, Ryo Kato, Mamoru Tomita: Activation of Bacillus spp. Spores by Hydrostatic Pressure, Journal of the Brewing Society of Japan, Vol. 50 (No. 2), 104110, 1995

### SUMMARY OF INVENTION

### Technical Problem

Methods using microbes having the ability to degrade pollutants have been developed so far as methods for decontaminating environments polluted with petroleum-related materials. However, all of these microbes have low pressure resistance and make efficient construction difficult for construction that requires high-pressure injection deep in the ground. In many cases, the survival of the microbes is influenced by temperatures, etc. of the environments and disadvantageously requires extreme caution in conditions of preservation or the environments (temperature, pressure, pH, etc.) for use.

### Solution to Problem

The inventor of the present application has conducted diligent studies to develop an approach of solving such technical difficulty, and completed the invention of the present application.

The inventor has conducted diligent studies on the process using spores (endospores) of bacteria as organisms resistant to environmental loads such as temperature, pH, and pressure [Non Patent Literature 4: Kenji Nishi, Ryo Kato, Mamoru Tomita: Activation of Bacillus spp. Spores by Hydrostatic Pressure, Journal of the Brewing Society of Japan, Vol. 50 (No. 2), 104110, 1995] as a method that solves the difficulty of the conventional bioaugmentation method described above and can solve limited environmental conditions for preservation or use, and completed the invention of the present application.

For information, microbes that have been used in the conventional process die completely at a temperature on the order of 100° C. or at a pressure on the order of 5 MPa. By contrast, strains developed by the invention of the present application are strains that neither die at 100° C. nor die at a pressure of 200 MPa.

Specifically, bacteria degrading petroleum-related materials (hydrocarbons) under aerobic conditions were selected from strains able to form endospores and to survive even at a temperature of 70° C. A plurality of bacteria obtained by search successfully exerted a function of degrading even macromolecular hydrocarbon compounds (hydrocarbons having 35 or more carbon atoms) through their simultaneous action. A basic technology invention of the present application has been completed.

The present invention provides the following aspects.

A method for degrading a petroleum-related material, comprising using a microbe of the genus Bacillus.

The method for degrading a petroleum-related material according to [1], wherein the microbe is used in combination with a component for promoting the proliferation of the microbe.

The method for degrading a petroleum-related material according to claim 1, wherein the microbe is used in combination with a biomass material.

The method for degrading a petroleum-related material according to [1], wherein a spore of a bacterium of the genus Bacillus is used.

The method for degrading a petroleum-related material according to [1], wherein the bacterium of the genus Bacillus is a bacterium of the genus Bacillus having high gene homology to a member selected from the group consisting of Bacillus siamensis, Bacillus amyloliquefaciens, and Bacillus velezensis.

The method for degrading a petroleum-related material according to [1], wherein a macromolecular (having 30 or more carbon atoms) petroleum-related material in soil is degraded.

The method for degrading a petroleum-related material according to [1], wherein a cell or a spore of a Bacillus sp. af-1 strain (Accession No. NITE P-02735) and a cell or a spore of a Bacillus sp. af-5 strain (Accession No. NITE P-02736) are used in combination.

A method comprising degrading and removing a hydrocarbon in water using a bacterium of the genus Bacillus.

A method for biochemical degradation of hydrocarbons, comprising using a microbe of the genus Bacillus.

A bacterium of the genus Bacillus or a spore thereof, wherein the bacterium isbeing a bacterium of the genus Bacillus having high gene homology to a member selected from the group consisting of Bacillus siamensis, Bacillus amyloliquefaciens, and Bacillus velezensis, and has degradative activity against a petroleum-related material, or a spore thereof.

A microbe selected from the group consisting of a Bacillus sp. af-1 strain (Accession No. NITE P-02735) and a progeny thereof, a Bacillus sp. af-5 strain (Accession No. NITE P-02736) and a progeny thereof, and spores (endospores) of these strains and progeny.

An environmental pollutant removing agent comprising a cell or a spore of a Bacillus sp. af-1 strain (Accession No. NITE P-02735) and a cell or a spore of a Bacillus sp. af-5 strain (Accession No. NITE P-02736) as active ingredients.

### Advantageous Effects of Invention

The present invention provides a microbe having resistance to even high temperatures or high pressures while having excellent ability to degrade petroleum-related materials. The present invention is capable of solving the problems of limited environmental conditions for preservation or use of environmental pollutant removing agents, and can provide a bioremediation technique, particularly, a bioaugmentation technique, which employs an organism resistant to environmental loads such as temperature, pH, and pressure, is highly useful, and utilizes the excellent ability to degrade petroleum-related materials. For example, strains developed by the invention of the present application are strains that neither die at 100° C. nor die at a pressure of 200 MPa. These strains have features of being able to form endospores and to survive even at high temperatures and degrading petroleum-related materials (e.g., hydrocarbons) under aerobic conditions. A plurality of strains can exert an excellent function and ability to degrade even macromolecular hydrocarbon compounds (hydrocarbons having 35 or more carbon atoms, etc.) through their simultaneous action.

Other objects, features, advantages and aspects of the present invention will become apparent to those skilled in the art upon reading the following description. However, it should be understood that in the description of the present specification including the following description and the description of specific Examples, etc., only the preferred embodiments of the present invention are shown and described, simply by way of illustration. As will be realized by those skilled in the art, various changes and/or modifications can be made in the present invention on the basis of knowledge from the following description and other parts of the present specification, without departing from the intention and the scope disclosed in the present specification. All patent literatures and references cited herein are cited by way of illustration, and their contents should be interpreted as being incorporated herein by reference in their entirety.

### BRIEF DESCRIPTION OF DRAWINGS

FIG. 1 shows a photograph as a drawing substitute showing the manner of degradation of lubricating oil in culture solutions of sf-1 and sf-5 strains. Medium: 0.5% peptone, 0.5% yeast extracts, and 5% lubricating oil, adjusted with sodium hydroxide of pH 7.0. Culture conditions: 28° C., shake culture at 100 rpm, 36 hr. For a control, no inoculation was performed, and the other conditions were the same as above.

FIG. 2 shows a report from TechnoSuruga Laboratory Co., Ltd. as to an sf-1 strain found and isolated in Example 1.

FIG. 3 shows a report from TechnoSuruga Laboratory Co., Ltd. as to an sf-5 strain found and isolated in Example 1.

FIG. 4 shows a GC-FID analysis chart of a soil sample containing diesel oil by treatment with the sf-1 strain and the sf-5 strain used in combination.

FIG. 5 shows a GC-FID analysis chart of a soil sample containing lubricating oil by treatment with the sf-1 strain and the sf-5 strain used in combination.

FIG. 6 shows change in GC-FID analysis chart of hydrocarbons in a soil sample treated through reaction with the microbial materials of the present invention.

### DESCRIPTION OF EMBODIMENTS

Methods using microbes having the ability to degrade pollutants have been developed so far as methods for decontaminating environments polluted with petroleum-related materials. However, all of these microbes have low pressure resistance and make efficient injection difficult for construction that requires high-pressure injection deep in the ground. In many cases, the survival of the microbes is influenced by temperatures, etc. of the environments and disadvantageously requires extreme caution in conditions of preservation or the environments (temperature, pressure, pH, etc.) for use.

In the present invention, a method for solving such problems has been found as follows.

The present inventor has conducted diligent studies on the process using spores (endospores) of bacteria as organisms resistant to environmental loads such as temperature, pH, and pressure [Kenji Nishi, Ryo Kato, Mamoru Tomita: Activation of Bacillus spp. Spores by Hydrostatic Pressure, Journal of the Brewing Society of Japan, Vol. 50 (No. 2), 104110, 1995] as a method that can solve limited environmental conditions for preservation or use for the purpose of solving the difficulty of the conventional bioaugmentation method described above, and completed the invention of the present application.

For information, microbes that have been used in the conventional process die completely at a temperature on the order of 100° C. or at a pressure on the order of 5 MPa. By contrast, microbial materials developed by the invention of the present application are strains that neither die at 100° C. nor die at a pressure of 200 MPa.

In the present invention, bacteria degrading petroleum-related materials (hydrocarbons) under aerobic conditions were selected as the microbes used from strains able to form endospores and to survive even at a temperature of 70° C. A plurality of bacteria obtained by search successfully exerted a function of degrading even macromolecular hydrocarbon compounds (hydrocarbons having 35 or more carbon atoms) through their simultaneous action. A technology invention of the present application has been completed.

The microbe of the present invention belongs to the genus Bacillus and has the ability to degrade a petroleum-related material, for example, a hydrocarbon, under aerobic conditions. The microbe of the present invention, albeit having weak degradative activity against lubricating oil, may produce a material having a surface activating function and promote the degradation of lubricating oil by another microbe, and furthermore, its degradative activity against lubricating oil may be potentiated by another microbe producing a material having a surface activating function. Examples of the microbe of the present invention include microbes selected by culture using activity of degrading a petroleum product, for example, lubricating oil, as an index. Examples of such a microbe can include an sf-1 strain and an sf-5 strain isolated by the present inventor.

The microbe of the genus Bacillus of the present invention may include a strain that exhibits high gene homology (e.g., 99.6% homology) to a member selected from the group consisting of a Bacillus siamensis PD-A10^{T} strain, a Bacillus amyloliquefaciens NBRC 15535^{T} strain, and a Bacillus velezensis CR-50^{T} strain, but has higher gene homology than the homology from homology analysis on the nucleotide sequences of ribosomal DNA (16S rDNA-500) of the sf-1 and sf-5 strains, i.e., SEQ ID NO: 1 and SEQ ID NO: 2 of the sequence listing.

The microbe of the present invention also includes, in addition to the sf-1 strain and the sf-5 strain, microbes that exhibit given similarity to these strains. The microbes that exhibit given similarity refer to, for example, microbes similar in the nucleotide sequence of ribosomal DNA (16S rDNA-500) to these two strains. Specifically, the microbes that exhibit given similarity to the strains include microbes having a nucleotide sequence of ribosomal DNA (16S rDNA-500) homologous by 99.7% or more, preferably 99.8% or more, particularly preferably 99.9% or more, to the nucleotide sequence represented by SEQ ID NO: 1 or SEQ ID NO: 2.

Such microbes that exhibit given similarity to the strains can be isolated through the use of an approach described in Example 1 using degradative activity against a petroleum-related material, for example, volatile oil, as an index, or from a soil sample using the nucleotide sequence represented by SEQ ID NO: 1 or 2 as an index.

A usual culture method is used for proliferating the microbe of the present invention by culture. The culture can be suitably performed under aerobic conditions. For the culture, a medium containing a carbon source, a nitrogen source, or the like assimilable or utilizable by the microbe is used. The carbon source may be any source assimilable or utilizable by the microbe. Examples thereof include sugars or carbohydrates such as glucose, sucrose, fructose, molasses, starch, soluble starch, starch hydrolysates, and dextrin, and paraffins, and further include organic acids such as acetic acid, citric acid, butyric acid, fumaric acid, and benzoic acid, alcohols such as methanol, ethanol, butanol, and glycerin, fatty acids such as oleic acid and stearic acid and esters thereof, and oils and fats such as soybean oil, rapeseed oil, and lard oil. These carbon sources can be used singly or as a mixture.

The nitrogen source may be any source utilizable by the microbe. Examples thereof include ammonia, ammonium salts such as ammonium chloride, ammonium sulfate, ammonium nitrate, ammonium acetate, and ammonium phosphate, nitrates such as sodium nitrate and potassium nitrate, urea, peptone, casamino acids, corn steep liquor, corn gluten meal, casein hydrolysates, bran, yeast extracts, dry yeasts, various fermented microbial cells and digestion products thereof, soybean flour, soymeal and soymeal hydrolysates, cottonseed flour, meat extracts, and other organic or inorganic nitrogen-containing materials. These nitrogen sources can be used singly or as a mixture. The medium may be appropriately supplemented with optional nutrients such as inorganic salts, minerals, vitamins, and trace metal salts.

Examples of the material to be added as an inorganic salt to the medium include phosphate, magnesium salt, calcium salt, iron salt, and other optional trace metal salts. A vitamin or the like may be added in an appropriate amount as a nutrient source for promoting the proliferation of the microbe of the present invention. In addition, a material generally used in the art can be appropriately selected and used. The microbe of the present invention is inoculated to a medium or the like containing an organic material, an inorganic salt, a nitrogen source, and other nutrient sources, and cultured under aerobic conditions by, for example, a static culture method, a shake culture method, or a deep-aerated stirring culture method.

Temperature conditions for the culture are set to the range of growing temperatures, preferably, the optimum growing temperatures, of the microbe used. The culture temperature can be set to, for example, 20 to 30° C., preferably 25° C. The pH of the medium can be set to the range of 6.5 to 7.5.

The culture time differs depending on the amount or type of a nutrient source and may be selected such that sufficient proliferation can be achieved. The culture time may be, for example, several minutes, several hours, or several days.

The microbe of the present invention can be utilized in the purification of environments polluted with petroleum-related materials. Specifically, the present invention provides an environmental pollutant removing agent comprising the microbe as an active ingredient. In this context, the “environment” includes soil, ocean, lake, river, and groundwater as well as wastewater, etc. The environment may be meant to further include wastes, for example, industrial wastes, household wastes, factory wastes, industrial wastewater, etc. The environment is purified by spraying or injecting a culture solution of the microbe cultured under the conditions described above, a spore (endospore)-containing liquid of the microbe, or a dry powder obtained by the freeze-drying treatment of the microbe or spores thereof to the polluted environment. In this operation, a plurality of microbes may be used in combination, or the microbe may be mixed or granulated with a component assisting in its proliferation, or inorganic salts and thereby formulated into a powder, granules, or the like, which can then be sprayed to the polluted environment. The amount of the microbe used in the treatment can be arbitrarily set according to the pollution situation of soil and seawater, etc. Polluted wastewater is similarly purified by mixing the culture solution or dried microbial cells with the polluted wastewater, followed by culture under aerobic conditions.

The “petroleum-related material” targeted by the present inventio may include liquid fossil fuel and may encompass, for example, crude oil, petroleum products produced by the purification and separation of crude oil, for example, gasoline, kerosene, light oil, heavy oil, lubricating oil, engine oil, diesel oil, paraffin, tar, and asphalt, and further, their components, i.e., saturated hydrocarbons such as linear alkane, branched alkane, cycloalkane, and alkyl cycloalkane, unsaturated aliphatic hydrocarbons, aromatic hydrocarbons such as benzene, toluene, naphthalene, phenanthrene, and anthracene, their hydrocarbon derivatives, sulfur compounds, nitrogen compounds, and oxygen compounds. The petroleum-related material may include a material that is generated by a petroleum product-derived organic synthesis technique and has adverse effects on environments.

The present invention also relates to a technique of obtaining much better degradation performance of petroleum-related materials by using in combination two or more of the microbes that exhibit resistance to high temperatures, high pressures, etc. For example, use of a cell or a spore of a Bacillus sp. af-1 strain (Accession No. NITE P-02735) and a cell or a spore of a Bacillus sp. af-5 strain (Accession No. NITE P-02736) in combination as active ingredients can favorably degrade even a macromolecular hydrocarbon heretofore regarded as being difficult to degrade, for example, a macromolecular hydrocarbon of approximately C₃₀. The present invention further provides a technique using the microbe in combination with a biomass material assimilable or utilizable by the microbe, and a biomass material that exhibits growth promoting activity. The use in combination with these biomass materials is capable of producing much better degradation performance of petroleum-related materials, and is also capable of efficiently decontaminating environments. Examples of the biomass materials include biomass materials of various organism origins. For example, nutrient sources for use in the culture described above may be included therein, and biomass materials in various forms may be used. The biomass materials can be selected, for use, from among those known to be biologically utilizable in the art or in other fields. Examples thereof include waste biomasses, unused biomasses, and resource crop-derived biomasses. Examples of the waste biomasses include discarded paper, pulping waste liquors, livestock excrements, food wastes, wood chips derived from construction, remaining materials from lumber mills, sawdust, branches and leaves of tress, grass, sewage sludge, and sewage organic materials. Examples of the unused biomasses include rice straw, wheat straw, chaffs, unused portions of agricultural crops, seagrass, and planktons. Examples of the resource crop-derived biomasses include sugar cane and corn. In the present invention, use in combination of substances, such as surfactants, which have activity of emulsifying oil components, or use in combination of microbes producing such components is also advantageous. For information, the Bacillus sp. af-1 strain produces a substance having a surface activating function.

Hereinafter, the present invention will be specifically described with reference to Examples. These Examples are provided for reference of specific aspects thereof in order to merely illustrate the present invention. These examples illustrate certain specific aspects of the present invention, but do not intend to limit the scope of the invention disclosed in the present application. It should be understood that various embodiments based on the brief of the present specification can be carried out in the present invention.

All Examples were carried out or can be carried out by use of standard techniques, which are well known to and commonly used by those skilled in the art, unless otherwise specified in detail.

The sf-1 strain described in Example 1 mentioned later has been deposited and stored since Jun. 7, 2018 in National Institute of Technology and Evaluation (NITE) Patent Microorganisms Depositary (NPMD) (2-5-8 Kazusakamatari, Kisarazu-shi, Chiba 292-0818, Japan) (identification code: Bacillus sp. af-1, Accession No. NITE P-02735, date of receipt: Jun. 7, 2018). The sf-5 strain described in Example 1 mentioned later has been deposited and stored since Jun. 7, 2018 in National Institute of Technology and Evaluation (NITE) Patent Microorganisms Depositary (NPMD) (2-5-8 Kazusakamatari, Kisarazu-shi, Chiba 292-0818, Japan) (identification code: Bacillus sp. af-5, Accession No. NITE P-02736, date of receipt: Jun. 7, 2018).

### Example 1

### Obtainment of Strain Having Ability to Degrade Petroleum-Related Material and Qualitative Analysis of Degradative Activity Against Petroleum-Related Material

Strains having the ability to degrade petroleum-related materials were searched for by the following approach.

Specifically, 5 mL of a sterilized liquid (aqueous solution containing 0.5% peptone and 0.1% yeast extracts) and 0.5 mL to 1.0 mL of lubricating oil are placed in a glass test tube having a bore of 1.3 cm, and stirred.

A sample of a source of isolation was added in a small amount to the mixture used as a screening medium, and shake-cultured at 27° C.

In this culture solution, petroleum-related materials, when left standing, form a layer on the upper surface of the liquid so as to separate from a culture solution layer. The culture solution layer of a sample decreased in this petroleum layer by culture was repetitively transplanted to the screening medium, followed by the enrichment culture of microbes.

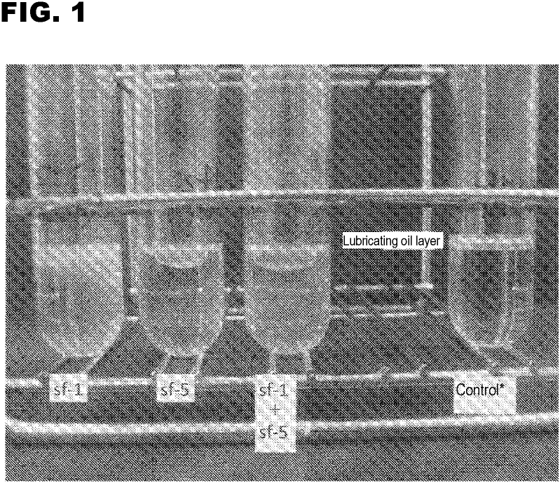

One example of this screening culture is shown in the photograph of FIG. 1. Strains regarded as having degradative activity against lubricating oil were collected by this approach. For information, studies related to the present invention discovered the formation of globular liquid foams containing petroleum-degrading microorganisms (see the photograph of FIG. 1), as shown in this Example. This was used as an index to enable petroleum-degrading microorganisms to be easily detected.

In this approach, soil sampled from Sakai-shi, Osaka, Japan was searched for microbes having activity of degrading petroleum (lubricating oil). Two bacterial strains (referred to as an sf-1 strain and an sf-5 strain) suitable for the purpose were isolated from the sludge of the city. The sf-1 strain, albeit having weak degradative activity against lubricating oil, produced a material having a surface activating function and promoted the degradation of lubricating oil by the sf-5 strain.

### Example 2

The present invention relates to a method for decontaminating petroleum pollution using both of the sf-1 strain and/or the sf-5 strain. In Examples given below, the following [Measurement method for degradative activity] against petroleum-related materials was used unless otherwise specified.

Activity of decontaminating soil polluted with petroleum-related materials according to the present invention was measured (hereinafter, referred to as activity measurement) according to the following approach.

The soil product used was collected from farm land created by the ground leveling of forests in Osaka, Japan. The sample of petroleum-polluted soil used was soil collected from approximately 1 meter deep from the surface of the farm land, and prepared by allowing diesel oil and/or lubricating oil to be adsorbed to the soil. Both the diesel oil and the lubricating oil used were commercial products sold in Japan.

A mixture of spores of the sf-1 strain and the sf-5 strain (M-U) and a material inducing and promoting the germination and proliferation of the spores in M-U (A-U) were added thereto and thoroughly stirred to prepare a sample for activity measurement.

The activity measurement was performed by the following procedures: the sample for activity measurement was reacted at 27° C. Petroleum-related materials in the sample were extracted by the approach shown below in [Petroleum-related material extraction method], and analyzed by a weight method of measuring change in the weight of the extracts and gas chromatography (GC-FID) under the conditions shown in Table 1.

TABLE 1

Conditions for gas chromatography (GC-FID)

| | |
|---|---|
| Gas chromatography apparatus | GC-17A (Shimadzu Corp.) |
| Column | DB-1HT (Agilent Technologies, Length: 30 m Diam: 0.320 µm, Film: 0.10 µm) |
| Carrier gas | Helium gas (flow rate: 3 mL/min) |
| Oven temperature | 50° C. (10 min) -10° C./min. -350° C./10 min |
| Injection volume | 1.0 µl |
| Injection mode | split (15:1) |
| Injection temperature | 350° C. |
| Detector temperature | 350° C. |
| Flow rate | 3.01 ml/min |

### Measurement Method for Degradative Activity

The activity of decontaminating soil polluted with petroleum-related materials (and/or a polluted soil preparation) according to the present invention was measured (hereinafter, referred to as activity measurement) according to the following approach.

Diesel oil or lubricating oil was added to the soil preparation at a predetermined weight ratio and thoroughly stirred using a blender to prepare a petroleum-polluted soil sample. M-U and A-U were added thereto and thoroughly stirred using a blender to prepare a sample for activity measurement.

The activity measurement was performed by the following procedures: the sample for activity measurement was reacted at 27° C. Petroleum-related materials in the sample were extracted by the approach shown below in [Petroleum-related material extraction method], and analyzed by a weight method of measuring change in the weight of the extracts and gas chromatography (GC-FID) under the conditions shown in Table 1.

### Petroleum-Related Material Extraction Method

Standard procedures for the extraction of petroleum-related materials from the soil preparation are as follows: (1) 5 g of the soil sample was placed in a 50 mL Falcon tube; (2) 15 g of Na₂SO₄ and 15 mL of n-hexane were added thereto and stirred to extract petroleum-related materials; (3) the liquid extracts were collected by filtration, and this operation was repeated three times to prepare liquid extracts; and (4) the resulting liquid extracts were concentrated under reduced pressure to remove n-hexane, and the petroleum-related materials were collected and weighed to measure the content of the petroleum-related materials. The degradative activity of M-U and A-U against the petroleum-related materials was measured by this approach.

The content of the petroleum-related materials was also measured by a common analysis method based on gas chromatography under the conditions shown in Table 1.

### Example 3

### Identification of Strain Sf-1 and Strain Sf-5

Both the sf-1 strain and the sf-5 strain found and isolated in Example 1 were aerobic bacteria and were gram-positive rods forming spores. These properties indicate that the bacteria are taxonomically classified into the genus Bacillus.

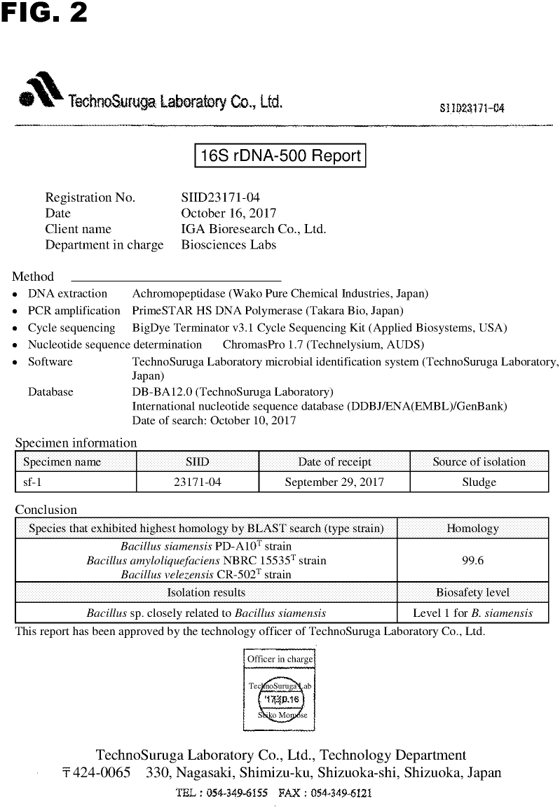

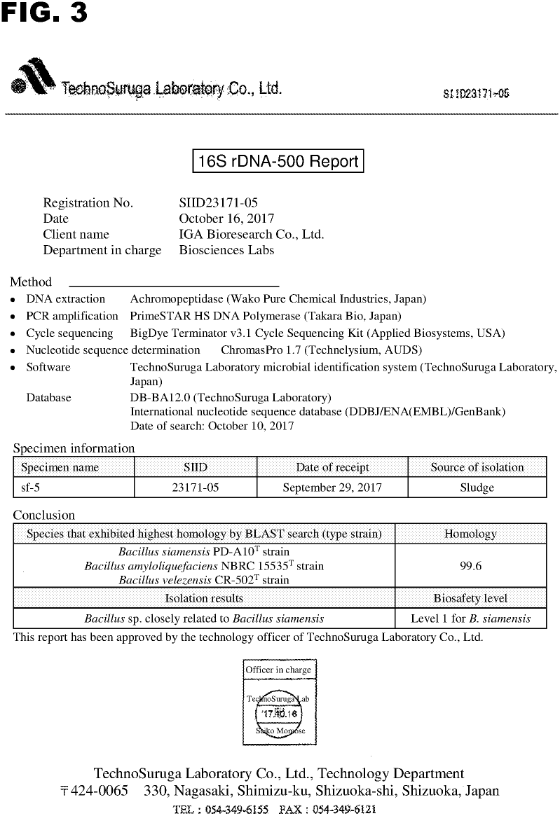

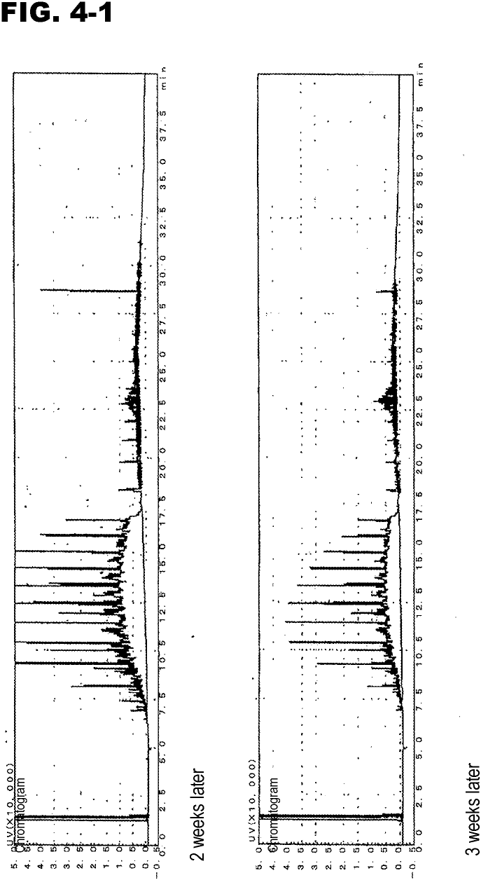

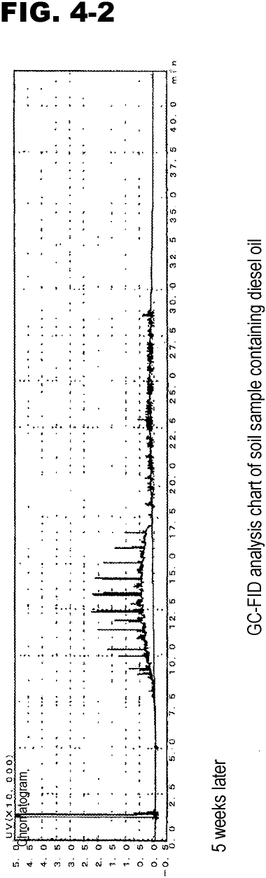

From homology analysis on ribosomal DNA (16S rDNA-500) (FIGS. 2 and 3), these two strains were both identified as Bacillus sp. closely related to Bacillus siamensis (percent homology: 99.6%).

The nucleotide sequences of 16S rDNA-500 (from the reports of TechnoSuruga Laboratory Co., Ltd.) extracted from the strain sf-1 and the strain sf-5 are shown below.

### Nucleotide Sequence of DNA of Sf-1 Strain [SEQ ID NO: 1 of the Sequence Listing

[Formula 1]

(Nucleotide sequence of DNA of sf-1 strain)GACGAAC GCTGGCGGCGTGCCTAATACATGGAAGTCGAGCGGACAGATGGGAGCTTG CTCCCTGATGTTAGCGGCGGACGGGTGAGTAACACGTGGGTAACCTGCCT GTAAGACTGGGATAACTCCGGGAAACCGGGGCTAATACCGGATGGTTGTC TGAACCGCATGGTTCAGACATAAAAGGTGGCTTCGGCTACCACTTACAGA TGGACCCGCGGCGCATTAGCTAGTTGGTGAGGTAACGGCTCACCAAGGCG AGGATGCGTAGCGGACCTGAGAGGGTGATCGGCCACACTGGGACTGAGAC ACGGCCCAGACTCCTACGGGAGGCAGCAGTAGGGAATCTTCCGCAATGGA CGAAAGTCTGACGGAGCAACGCCGCGTGAGTGATGAAGGTTTTCGGATCG TAAAGCTCTGTTGTTAGGGAAGAACAAGTGCCGTTCAAATAGGGCGGCAC CTTGACGGTACCTAACCAGAAAGCCACGGCTAACTACGTGC

### Nucleotide Sequence of DNA of sf-5 Strain [SEQ ID NO: 2 of the Sequence Listing

[Formula 2]

(Nucleotide sequence of DNA of sf-5 strain)GAGGAAC GCTGGCGGCGTGCCTAATACATGCAAGTCGAGCGGACAGATGGGAGCTTG CTCCCTGATGTTAGCGGCGGACGGGTGAGTAACACGTGGGTAACCTGCCT GTAAGACTGGGATAAGTCGGGGAAACCGGGGCTAATACCGGATGGTTGTC TGAACCGCATGGTTCAGACATAAAAGGTGGCTTCGGCTACCACTTACAGA TGGACCCGCGGCGCATTAGCTAGTTGGTGAGGTAACGGCTCACCAAGGCG ACGATGCGTAGCCGACCTGAGAGGGTGATCGGCCACACTGGGGACTGAGA CACGGCCCAGACTCCTACGGGAGGCAGCAGTAGGGAATCTTCCGCAATGG ACGAAAGTCTGACGGAGCAACGCCGCGTGAGTGATGAAGGTTTTCGGATC GTAAAGCTCTGTTGTTAGGGAAGAACAAGTGCCGTTCAAATAGGOGGGGA CCTTGACGGTACCTAACCAGAAAGCCACGGCTAACTACGTGC

Species (type strains) that exhibited the highest homology by BLAST search were a Bacillus siamensis PD-A10^{T} strain, a Bacillus amyloliquefaciens NBRC 15535^{T} strain, and a Bacillus velezensis CR-50^{T} strain. As a result of comprehensive discussion, these strains were identified as Bacillus sp. closely related to Bacillus siamensis (reported from TechnoSuruga Laboratory Co., Ltd.; FIGS. 2 and 3). For information, Bacillus siamensis is a bacterial species often separated from Piukem (salted freshwater crabs originated in Thailand), and is a highly safe strain evaluated as a biosafety level (FIGS. 2 and 3; based on the biosafety guidelines of Japanese Society for Bacteriology).

From these facts, the sf-1 strain and the sf-5 strain are evaluated as very suitable microbes for use in the decontamination (bioaugmentation) of petroleum pollution using a large amount of microbial cells. In animal tests using mice, these strains were confirmed to be negative both to oral acute toxicity and to delayed hypersensitivity reaction.

### Example 4

### Degradation of Diesel Oil and Lubricating Oil By Use of sf-1 Strain and sf-5 Strain in Combination

All the soil preparations used in the present invention were collected from farm land created by the ground leveling of forests in Japan. The microbial material used was a powder containing 10⁷ spores each of the sf-1 strain and the sf-5 strain per g (hereinafter, referred to as M-U).

A-U was further developed as a material having germinating and proliferative activity against the microbial material. The component of A-U is not limited by its composition, regardless of being a single substance or a mixture, as long as the material has activity of germinating spores and proliferative activity against the strains. For example, a mixture of a carbohydrate, a protein, yeast extracts, a nucleic acid, phosphoric acid, and potassium, calcium, and magnesium salts was used, though these components can be used singly or as a mixture. Exemplary A-U of the present invention is a mixture containing 2% glucose, 0.5% peptone and 0.3% yeast extracts.

Hereinafter, 100 g of a soil preparation having the composition shown in Table 2 was placed in a 500 mL plastic container, and after cotton plugging, reacted at 27° C., followed by the analysis of diesel oil and lubricating oil to evaluate the effect of M-U.

TABLE 2

Conditions for evaluation test on activity of decontaminating soil polluted with petroleum product by microbial material

| Reactant symbol | Petroleum product content in soil | Amount added [%(w/w)] | | |
|---|---|---|---|---|
| | | M-U | A-U | Water |
| 1-1 | 5,000 ppm diesel oil | 5 | 5 | 30 |
| 1-2 | 5,000 ppm diesel oil | 10 | 5 | 25 |
| 1-3 | 5,000 ppm lubricating oil | 5 | 5 | 30 |
| 1-4 | 5,000 ppm lubricating oil | 10 | 5 | 25 |

As a result, it is evident from the comparison between the results about 1-1 and 1-2 or about 1-3 and 1-4 in Tables 3 to 6 that the degradation rate of diesel oil or lubricating oil by the former is larger than that by the latter.

TABLE 3

Change in content of diesel oil in reactant 1-1

| Reaction time (d) | Diesel oil concentration (ppm) |
|---|---|
| 7 | 4,700 |
| 14 | 4,100 |
| 21 | 3,900 |
| 35 | 3,600 |

TABLE 4

Change in concentration of diesel oil in reactant 1-2

| Reaction time (d) | Diesel oil concentration (ppm) |
|---|---|
| 7 | 2,600 |
| 14 | 1,650 |
| 21 | 1,530 |
| 35 | 1,160 |

TABLE 5

Change in concentration of lubricating oil in reactant 1-3

| Reaction time (d) | Lubricating oil concentration (ppm) |
|---|---|
| 7 | 4,300 |
| 14 | 4,100 |
| 21 | 3,950 |
| 35 | 2,940 |

TABLE 6

Change in concentration of lubricating oil in reactant 1-4

| Reaction time (d) | Lubricating oil concentration (ppm) |
|---|---|
| 7 | 4,680 |
| 14 | 1,450 |
| 21 | 1,280 |
| 35 | 1,100 |

These results demonstrated that the amount of the microbe (M-U) injected correlates with the amount of diesel oil or lubricating oil removed, and demonstrated that the strains isolated by the technique of the present invention are effective for the removal of diesel oil pollution.

### Example 5

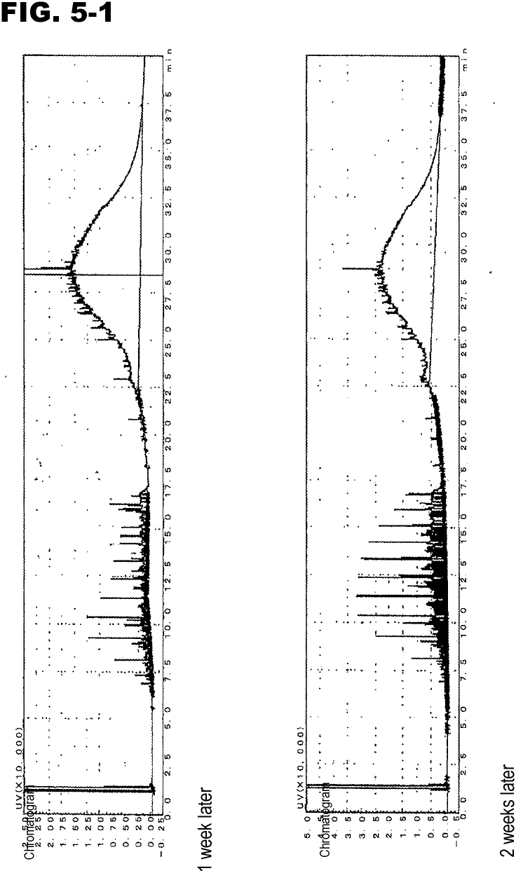

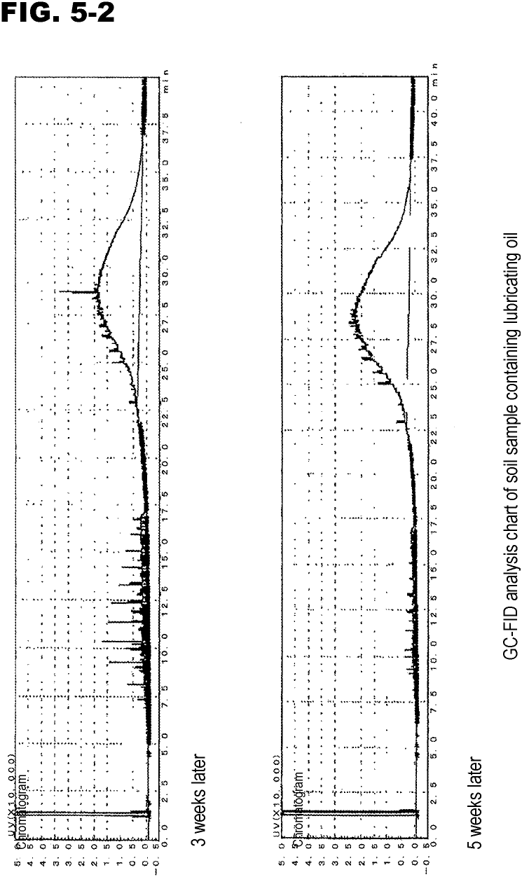

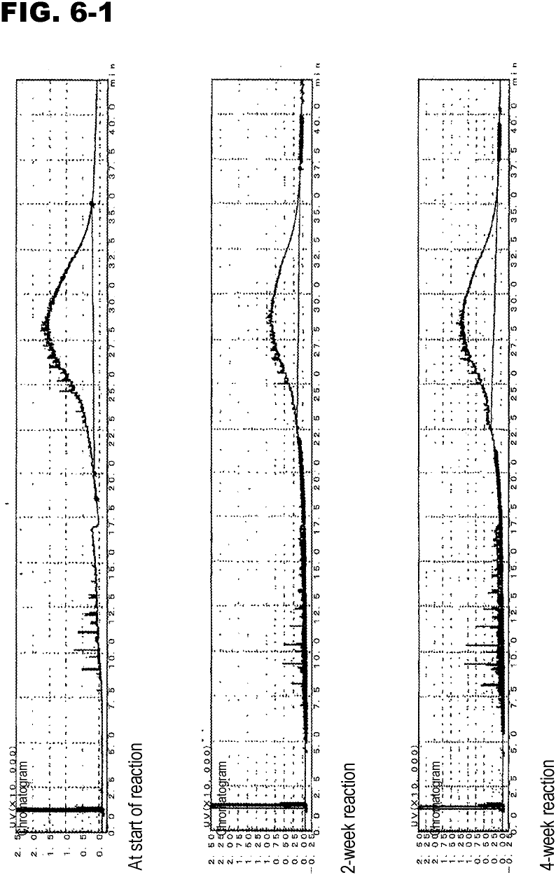

1-2 and 1-4 under the conditions shown in Table 2 shown in Example 4 were studied for the state of degradation of diesel oil or lubricating oil by GC-FID (gas chromatograph). The results are shown in FIGS. 4 and 5 by comparison. From these results, the microbial degradation of lubricating oil, which has heretofore been reported to be difficult in most cases, proceeded at a given rate in the method of the present invention, and the degradation reaction correlated with the amount of M-U injected, suggesting that the method can be exploited in the decontamination of lubricating oil.

From the results of analysis by GC-FID, it was revealed that this method can degrade macromolecular hydrocarbons of approximately C₃₀ (the number of carbon atoms: 30; the largest peak portion in the chromatogram). According to the previous findings, the microbial degradation of macromolecular hydrocarbons of approximately C₃₀ is not easy. Therefore, it can be concluded that the technique of the present invention is very valuable.

### Example 6

A lubricating oil decontamination test was conducted under conditions where the amount of soil was increased. Specifically, lubricating oil was added at 10,000 ppm to 3 kg of a soil sample and thoroughly stirred using a rotary drum-type mixer. Further, 60 g of M-U, 90 g of A-U and 750 mL of water were added thereto and thoroughly stirred to prepare a sample for decontamination. This sample was placed in a wood box (14 m × 13 cm × 16 cm), and the upper surface of the sample was covered with cotton cloth moistened with water. This wood box was placed in an upper stage of a thermostat having a tray containing water in a lower stage, and reacted at 25° C.

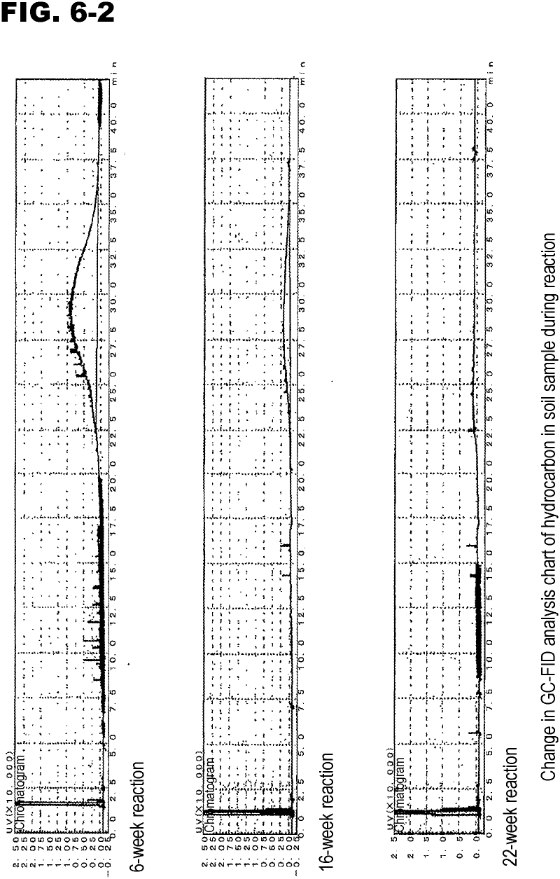

During the reaction, soil was collected from 5 cm deep from the sample surface at 1-week intervals, and the concentration of petroleum-related materials was measured. The measurement results are shown in Table 7 and FIG. 6. Lubricating oil became undetectable in the soil by the 22-week reaction. These results demonstrated the effectiveness of the present invention for the decontamination of soil polluted with petroleum-related materials.

TABLE 7

Change in Lubricating oil concentration in reacted soil (GC-FID analysis value)

| Reaction time (weeks) | Lubricating oil concentration (ppm) |
|---|---|
| 0 | 6,780 |
| 2 | 3,140 |
| 4 | 3,900 |
| 6 | 3,350 |
| 16 | 1,210 |
| 22 | Undetectable |

### INDUSTRIAL APPLICABILITY

In the present invention, a microbe has been found which is excellent in resistance to high temperatures or high pressures and also exhibits excellent degradative activity against macromolecular (having 30 or more carbon atoms) hydrocarbons or the like. The petroleum-related material decontamination technique according to the present invention is capable of injection deep in the ground and is a practical technique that can be utilized in the bioremediation of petroleum pollution. According to the present invention, use of the microbe excellent in pressure resistance permits efficient construction in construction that requires high-pressure injection deep in the ground, and is capable of advantageously decontaminating environments polluted with petroleum-related materials. The present invention can contribute to the development of a safe and inexpensive approach of purifying land or groundwater polluted with harmful materials.

It is obvious that the present invention can be carried out in a manner other than the description mentioned above and the particular description of Examples. Many changes and modifications can be made in the present invention in light of the instruction mentioned above. Thus, such changes and modifications are included in the scope of the accompanying claims of the present invention.

### Accession No

Bacillus sp. af-1 strain: NITE P-02735
Bacillus sp. af-5 strain: NITE P-02736

### Free Text of Sequence Listing

SEQ ID NO: 1, 16S-rDNA

SEQ ID NO: 2, 16S-rDNA

[image]

1. A method for degrading a petroleum-related material, comprising using a microbe of the genus Bacillus.

2. The method for degrading a petroleum-related material according to claim 1, wherein the microbe is used in combination with a component for promoting the proliferation of the microbe.

3. The method for degrading a petroleum-related material according to claim 1, wherein the microbe is used in combination with a biomass material.

4. The method for degrading a petroleum-related material according to claim 1, wherein a spore of a bacterium of the genus Bacillus is used.

5. The method for degrading a petroleum-related material according to claim 1, wherein the bacterium of the genus Bacillus is a bacterium of the genus Bacillus having high gene homology to a member selected from the group consisting of Bacillus siamensis, Bacillus amyloliquefaciens, and Bacillus velezensis.

6. The method for degrading a petroleum-related material according to claim 1, wherein a macromolecular (having 30 or more carbon atoms) petroleum-related material in soil is degraded.

7. The method for degrading a petroleum-related material according to claim 1, wherein a cell or a spore of a Bacillus sp. af-1 strain (Accession No. NITE P-02735) and a cell or a spore of a Bacillus sp. af-5 strain (Accession No. NITE P-02736) are used in combination.

8. A method comprising degrading and removing a hydrocarbon in water using a bacterium of the genus Bacillus.

9. A method for biochemical degradation of hydrocarbons, comprising using a microbe of the genus Bacillus.

10. A bacterium of the genus Bacillus or a spore thereof, wherein the bacterium is a bacterium of the genus Bacillus having high gene homology to a member selected from the group consisting of Bacillus siamensis, Bacillus amyloliquefaciens, and Bacillus velezensis, and has degradative activity against a petroleum-related material.

11. A microbe selected from the group consisting of a Bacillus sp. af-1 strain (Accession No. NITE P-02735) and a progeny thereof, a Bacillus sp. af-5 strain (Accession No. NITE P-02736) and a progeny thereof, and spores (endospores) of these strains and progeny.

12. An environmental pollutant removing agent comprising a cell or a spore of a Bacillus sp. af-1 strain (Accession No. NITE P-02735) and a cell or a spore of a Bacillus sp. af-5 strain (Accession No. NITE P-02736) as active ingredients.


SEQUENCE LISTING (2 sequences)

SEQ ID NO: 1 - DNA, 498 bp, Bucillus sp. sf-1
  source (1)..(498): 16S-rDNA
gacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgc

SEQ ID NO: 2 - DNA, 498 bp, Bucillus sp. sf-5; identical to SEQ ID NO: 1
  source (1)..(498): 16S-rDNA